(19) United States
(12) Patent Application Publication
(10) Pub. No.: US 2009/0081159 A1
(43) Pub. Date: Mar. 26, 2009

(54) COMPOSITIONS AND METHODS FOR DETECTING AND TREATING OPHTHALMIC DISORDERS

(75) Inventor: David N. Zacks, Ann Arbor, MI (US)

Correspondence Address: Casimir Jones, S.C., 440 Science Drive, Suite 203, Madison, WI, 53711, US

(73) Assignee: THE REGENTS OF THE UNIVERSITY OF MICHIGAN, Ann Arbor, MI (US)

(21) Appl. No.: 12/234,195

(22) Filed: Sep. 19, 2008

Related U.S. Application Data
(60) Provisional application No. 60/974,333, filed on Sep. 21, 2007.

(51) Int. Cl. A61K 38/20; C12Q 1/02; A61P 27/02

(52) U.S. Cl. 424/85.2; 435/29

(57) ABSTRACT

The present invention relates to compositions and methods for the detecting, treating, and conducting research on ophthalmic disorders associated with photoreceptor cell death and/or retinal insult. In particular, the present invention provides compositions and methods for increasing IL-6 expression and/or activity (e.g., exogenous IL-6), activating IL-6 receptors (e.g., IL-6R, sIL6-R), activating pathway related compounds (e.g., STAT, FLIP), and/or activating related pathways (e.g., JAK/STAT pathway, TGF-β pathway, Ahr pathway) in the diagnosis, treatment, and conducting research of ophthalmic disorders associated with photoreceptor cell death and/or retinal insult (e.g., retinal detachment).

### CROSS-REFERENCE TO RELATED APPLICATION

This application claims priority to pending U.S. Provisional Patent Application Ser. No. 60/974,333, filed Sep. 21, 2007, hereby incorporated by reference in its entirety.

### STATEMENT REGARDING FEDERALLY SPONSORED RESEARCH OR DEVELOPMENT

This invention was made with government support under Grant Nos. EY014705 and EY007003 awarded by the National Eye Institute. The government has certain rights in the invention.

### FIELD OF THE INVENTION

The present invention relates to compositions and methods for the detecting, treating, and conducting research on ophthalmic disorders associated with photoreceptor cell death and/or retinal insult. In particular, the present invention provides compositions and methods for increasing IL-6 expression and/or activity (e.g., exogenous IL-6), activating IL-6 receptors (e.g., IL-6R, sIL6-R), activating pathway related compounds (e.g., STAT, FLIP), and/or activating related pathways (e.g., JAK/STAT pathway, TGF-β pathway, Ahr pathway) in the diagnosis, treatment, and conducting research of ophthalmic disorders associated with photoreceptor cell death and/or retinal insult (e.g., retinal detachment).

### BACKGROUND OF THE INVENTION

Next to central retinal artery occlusion and chemical burns to the eye, retinal detachment is one of the most time-critical encountered eye emergencies. Retinal detachment (RD) was first recognized in the early 1700s by de Saint-Yves, but clinical diagnosis remained elusive until Helmholtz invented the ophthalmoscope in 1851.

Retinal detachment refers to separation of the inner layers of the retina from the underlying retinal pigment epithelium (RPE, choroid) and choroid. The choroid is a vascular layer underneath the retina which provides metabolic and nutritional support required by the photoreceptors. The RPE is a monolayer of large branched pigment cells sandwiched between the choroid and the retina, which also participates in metabolic support of the photoreceptors. Separation of the sensory retina from the underlying RPE occurs by the following 3 basic mechanisms: 1) a hole, tear, or break in the neuronal layer allowing fluid from the vitreous cavity to seep in between and separate sensory and RPE layers (ie, rhegmatogenous RD); 2) traction from inflammatory or vascular fibrous membranes on the surface of the retina, which tether to the vitreous; and/or 3) exudation of material into the subretinal space from retinal vessels such as in hypertension, central retinal venous occlusion, vasculitis, or papilledema. RDs may be associated with congenital malformations, metabolic disorders, trauma (including previous ocular surgery), vascular disease, choroidal tumors, high myopia or vitreous disease, or degeneration (including age-related macular degeneration). Of the 3 types of retinal detachment, rhegmatogenous RD is the most common, deriving its name from rhegma, meaning rent or break. Vitreous fluid enters the break and separates the sensory retina from the underlying RPE, resulting in detachment. Exudative or serous detachments occur when subretinal fluid accumulates and causes detachment without any corresponding break in the retina. The etiologic factors are often tumor growth or inflammation. Tractional retinal detachment occurs as a result of adhesions between the vitreous gel and the retina. Centripetal mechanical forces cause the separation of the retina from the RPE without a retinal break. Advanced adhesion may result in the development of a tear or break. The most common causes of tractional RD are proliferative vitreoretinopathy, proliferative diabetic retinopathy, sickle cell disease, advanced retinopathy of prematurity, and penetrating trauma. Vitreoretinal traction increases with age, as the vitreous gel shrinks and collapses over time, frequently causing posterior vitreous detachments in approximately two thirds of persons older than 70 years.

Although 6% of the general population have retinal breaks, most of these are benign atrophic holes, which are without accompanying pathology and do not lead to retinal detachment. Incidence of retinal detachment is 1 in 15,000 population, with a prevalence of 0.3% in the United States. The annual incidence is approximately one in 10,000 or about 1 in 300 over a lifetime (see, e.g., Haimann M H, et al., Arch Ophthalmol February 1982; 100(2): 289-92; herein incorporated by reference in its entirety). Other sources suggest that the age-adjusted incidence of idiopathic retinal detachments is approximately 12.5 cases per 100,000 per year, or about 28,000 cases per year in the United States (see, e.g., Subramanian M L, et al., Int Ophthalmol Clin 2004; 44(4): 103-14; herein incorporated by reference in its entirety). Certain groups have higher prevalence than others. Patients with high myopia (>6 diopters), a condition that is more common in males than in females have a 5% risk; individuals with aphakia (ie, cataract removal without lens implant) have a 2% risk. Cataract extraction complicated by vitreous loss during surgery has an increased detachment rate to 10%. Estimates reveal that 15% of people with retinal detachments in one eye develop detachment in the other eye. Risk of bilateral detachment is increased (25-30%) in patients who have had bilateral cataract extraction.

The development of improved methods for detecting ophthalmic disorders (e.g., RD) and the related progression of such disorders are needed. In addition, new therapeutic treatments are needed for the treatment of ophthalmic disorders involving retinal detachment and are of broad interest to the medical community, as well as to the pharmaceutical and biotech industries. The present invention addresses these issues.

### SUMMARY OF THE INVENTION

The present invention relates to compositions and methods for the detecting, treating, and conducting research on ophthalmic disorders associated with photoreceptor cell death and/or retinal insult. In particular, the present invention provides compositions and methods for increasing IL-6 expression and/or activity (e.g., exogenous IL-6), activating IL-6 receptors (e.g., IL-6R, sIL6-R), activating pathway related compounds (e.g., STAT, FLIP), and/or activating related pathways (e.g., JAK/STAT pathway, TGF-β pathway, Ahr pathway) in the diagnosis, treatment, and conducting research of ophthalmic disorders associated with photoreceptor cell death and/or retinal insult (e.g., retinal detachment).

Experiments conducted during the development of embodiments for the present invention demonstrated that inhibition of IL-6 accelerates photoreceptor apoptosis in models of RD. As such, in some embodiments, detection of diminished (e.g., reduced, absent) IL-6 expression, IL-6 receptor activity (e.g., IL-6R, sIL6-R), and/or related pathway activity (e.g., FLIP expression, STAT expression) in an affected ocular region (e.g., RPE tissue, retinal tissue) following an ocular insult (e.g., retinal detachment) indicates an increased likelihood of photoreceptor cell death and potential visual impairment. In some embodiments wherein retinal detachment has occurred, detection of diminished (e.g., reduced, absent) IL-6 expression, IL-6 receptor activity (e.g., IL-6R, sIL6-R), and/or related pathway activity (e.g., FLIP expression, STAT expression) in the affected ocular region (e.g., RPE tissue, retinal tissue) indicates a diminishing of the IL-6 protective period following retinal insult and a likelihood of photoreceptor cell death and visual impairment. In addition, experiments conducted during the development of embodiments for the present invention demonstrated that the presence of IL-6 within the subretinal space following an event involving the retina (e.g., retinal trauma resulting in RD) prevented photoreceptor apoptosis in models of RD. As such, in some embodiments wherein retinal detachment has occurred, detection of IL-6 expression, IL-6 receptor activity (e.g., IL-6R, sIL6-R), and/or related pathway activity (e.g., FLIP expression, STAT expression) in the affected ocular region (e.g., RPE tissue, retinal tissue) indicates the likelihood that photoreceptor cell death is being hindered (e.g., prevented, reduced) by the presence of such IL-6 activity. In some embodiments wherein retinal detachment has occurred, detection of IL-6 expression, IL-6 receptor activity (e.g., IL-6R, sIL6-R), and/or related pathway activity (e.g., FLIP expression, STAT expression) in the affected ocular region (e.g., RPE tissue, retinal tissue) indicates the likelihood that the IL-6 protective period is in effect. In some embodiments, detection of a change (e.g., a reduction) in IL-6 expression, IL-6 receptor activity (e.g., IL-6R, sIL6-R), and/or related pathway activity (e.g., FLIP expression, STAT expression) between at least two or more time points in an affected ocular region (e.g., an area of retinal detachment) indicates a diminishing of the IL-6 protective period, and an increased risk for photoreceptor cell death and potential visual impairment.

In certain embodiments, the present invention provides methods for preventing photoreceptor apoptosis within ocular tissue comprising: administering a composition configured to promote IL-6 activity within the ocular tissue. In some embodiments, the ocular tissue comprises photoreceptor cells at risk for undergoing cellular apoptosis. In some embodiments, the ocular tissue comprises retinal tissue and/or retinal pigment epithelial tissue. In some embodiments, the retinal tissue is detached from the retinal pigment epithelial tissue. The methods are not limited to a particular type of subject (e.g., rat, cat, mouse, primate). In some embodiments, the subject is a living human. In some embodiments, the administration comprises direct administration of the composition within the ocular tissue.

In certain embodiments, the present invention provides methods for treating photoreceptor apoptosis in a subject experiencing photoreceptor apoptosis, comprising administering a pharmaceutical composition to the subject, wherein the pharmaceutical composition is configured to inhibit photoreceptor apoptosis within the ocular tissue. In some embodiments, the ocular tissue comprises retinal tissue and/or retinal pigment epithelial tissue. In some embodiments, the retinal tissue is detached from the retinal pigment epithelial tissue. The methods are not limited to a particular type of subject (e.g., rat, cat, mouse, primate). In some embodiments, the subject is a living human. In some embodiments, the subject has been diagnosed with retinal detachment.

In certain embodiments, the present invention provides methods for detecting photoreceptor apoptosis, comprising providing two or more samples from a subject diagnosed with retinal detachment, wherein the two or more samples comprise ocular tissue comprising retinal tissue and/or retinal pigment epithelial tissue, wherein each of the two or more samples are obtained at different time points, quantifying the amount of IL-6 activity in each of the two or more samples, identifying a risk of photoreceptor apoptosis based on the quantifying. In some embodiments, an increase in the quantified IL-6 activity indicates a decreased risk for photoreceptor apoptosis. In some embodiments, a decrease in the quantified IL-6 activity indicates an increased risk for photoreceptor apoptosis. In some embodiments, the two or more samples are two samples. In some embodiments, the second of the two or more samples is obtained more than 5 minutes (e.g., 10 minutes, 20 minutes, 30 minutes, 60 minutes, 2 hours, 5 hours, 12 hours, one day, two days, one week, etc.) after the first sample is obtained.

In certain embodiments, the present invention provides methods for identifying photoreceptor apoptosis inhibitors, comprising providing ocular tissue comprising photoreceptor cells experiencing cellular apoptosis, and a composition comprising an agent; administering the composition to the ocular tissue, quantifying the amount of IL-6 activity and photoreceptor apoptosis in the ocular tissue following administration of the composition to the ocular tissue, wherein the presence or increase of IL-6 activity and decreased photoreceptor apoptosis within the ocular tissue indicates the agent is a photoreceptor apoptosis inhibitor, wherein the absence or decrease of IL-6 activity and photoreceptor apoptosis within the ocular tissue indicates the agent is not a photoreceptor apoptosis inhibitor.

The present invention is not limited to a particular indicator of IL-6 activity. In some embodiments, IL-6 activity includes, but is not limited to, IL-6 activity, JAK/STAT pathway activity, TGF-β pathway activity, Ahr pathway activity, STAT expression and/or activity, FLIP expression and/or activity, endothelin 2 expression and/or activity, ceruloplasmin expression and/or activity, lipocalin 2 expression and/or activity, serpin A3N expression and/or activity, and fibroblast growth factor receptor-1 expression and/or activity.

In some embodiments, the methods of the present invention further comprise co-administration of one or more general ophthalmological pharmacological agents. Examples of general pharmacological agents include, but are not limited to, Lucentis (ranibizumab), Macugen (pegaptanib); Restasis (cyclosporine ophthalmic emulsion); Lumigan (bimatoprost ophthalmic solution); Travatan (travoprost ophthalmic solution); Valcyte (valganciclovir HCl); Betaxon; Quixin (levofloxacin); Rescula (unoprostone isopropyl ophthalmic solution) 0.15%; Visudyne (verteporfin for injection); Alamast; Zaditor; Alrex; Cosopt; Lotemax; Salagen Tablets; Viroptic; Vitravene Injection; Acular; Acular (ketorolac tromethamine ophthalmic solution) 0.5%; BSS Sterile Irrigating Solution; AK-Con-A (naphazoline ophthalmic); Alphagan (brimonidine); Ocuflox (ofloxacin opthalmic solution) 0.3%; OcuHist; Vistide (cidofovir injection); and Vitrasert Implant.

In some embodiments, the methods of the present invention further comprise co-administration of an additional procedure designed to treat ophthalmic disorders involving photoreceptor cell death, retinal detachment, and/or retinal degeneration. Examples of such procedures include, but are not limited to, cryopexy and laser photocoagulation, scleral buckle surgery, pneumatic retinopexy, and vitrectomy.

### BRIEF DESCRIPTION OF THE DRAWINGS

FIG. 1 shows Western blot analysis of activated forms of STAT1 and STAT3 in attached and detached retinas. The leftmost two lanes were generated using retinal tissue collected 1 day after detachment. The middle two lanes were generated using retinal tissue collected 3 days after detachment. The right-most two lanes were generated using retinal tissue collected 7 days after detachment. Retina-RPE separation was created in the left eye. Attached retina was obtained from the contralateral eye of the same animal. Equal loading was verified across all lanes.

FIG. 2 shows Terminal Deoxynucleotidyl Transferase Mediated dUTP Nick End Labeling (TUNEL) staining in wild-type versus IL-6^{-/-} mouse retinas harvested 3 days after detachment. A and B: Wild-type mice. C and D: IL-6^{-/-} mice. A, C: Fluorescein isothiocyanate (FITC) staining of TUNEL positive cells. B, D: FITC staining of TUNEL positive cells and propidium iodide (PI) staining of all cells. E: Graph summarizing TUNEL staining of wild-type and IL-6^{-/-} mouse retinas 3 days after detachment. Results are mean±SEM.*P=0.002; IL-6^{-/-} mice vs. wild-type mice.

FIG. 3 shows outer nuclear layer (ONL) cell counts in wild-type versus IL-6^{-/-} mice after retinal detachment. A-F: mouse retinal tissue stained with toludine blue. A-C: Wild-type. D-F: IL-6^{-/-}. A and D: Attached retina. B and E: Mouse retinas harvested 1 month after creation of the detachment. C and F: Mouse retinas harvested 2 months after creation of the detachment. G: Graph summarizing ONL cell counts/total retinal thickness in wild-type and IL-6^{-/-} mice 1 and 2 months after retinal detachment. Results are mean±SEM.*P=0.02; IL-6^{-/-} mice vs. wild-type mice at 1 month.

FIG. 4 shows TUNEL staining of detached rat retinas treated with IL-6-neutralizing antibody (NAB) or exogenous IL-6. A and B: Subretinal injection of vehicle only at the time of creation of the detachment. C and D: Subretinal injection of 0.1 g anti-rat IL-6 NAB at the time of creation of the detachment. E and F: Subretinal injection of 0.15 μg anti-human IL-6 NAB at the time of creation of the detachment. G and H: Subretinal injection of 15 ng exogenous human IL-6 at the time of creation of the detachment. A, C, E, G: Fluorescein isothiocyanate (FITC) staining of TUNEL positive cells. B, D, F, H: FITC staining of TUNEL positive cells and propidium iodide (PI) staining of all cells. I: Graph summarizing effects of subretinal anti-IL-6 NAB and exogenous IL-6 on TUNEL staining of rat retinas 3 days after detachment. Results are mean±SEM.*P=0.01; anti-human-IL-6 NAB vs. control and anti-human-IL-6 NAB vs. exogenous human IL-6.** P=0.03; anti-rat-IL-6 NAB vs. control and anti-rat-IL-6 NAB vs. exogenous human IL-6.

FIG. 5 shows the effects of IL-6-neutralizing antibody (NAB) compared with effects of exogenous IL-6 on rat retina outer nuclear layer (ONL) cell counts. A-G: rat retinal tissue stained with toluidine blue. A: Attached retina. B and C: Retina harvested 1 and 2 months after subretinal injection of vehicle only at the time of creation of the detachment, respectively. D and E: Retina harvested 1 and 2 months after subretinal injection of 0.15 μg anti-human IL-6 NAB at the time of creation of the detachment, respectively. F and G: Retina harvested 1 and 2 months after subretinal injection of 15 ng exogenous human IL-6 at the time of creation of the detachment, respectively. H: Graph summarizing effects of subretinal anti-IL-6 NAB and exogenous IL-6 on photoreceptor cell count of rat retinas 1 and 2 months after retinal detachment. Results are mean±SEM.*P=0.001; control vs. anti-IL-6 NAB. † P=0.05; control vs. exogenous IL-6. [symbol] P=0.02; control versus delayed exogenous IL-6. §P=0.02; control versus exogenous IL-6 reinjection at 1 month. The increase in cell counts between 4 weeks and 8 weeks for animals treated with exogenous IL-6 is within the accepted variability.

### DEFINITIONS

As used herein, the term “subject” refers to any animal (e.g., a mammal), including, but not limited to, humans, non-human primates, felines, rodents, and the like, which is to be the recipient of a particular treatment and/or procedure. Typically, the terms “subject” and “patient” are used interchangeably herein in reference to a human subject.

As used herein, the terms “ophthalmic disorder” or similar terms refer to diseases, disorders, and/or conditions associated with the eye. Examples include, but are not limited to, retinal detachment.

As used herein, the term “subject is suspected of having retinal detachment” or “subject is at risk for developing retinal detachment,” or similar terms, refer to a subject (e.g., human, mouse, rat, cat) that presents one or more signs or symptoms of retinal detachment or is being screened for retinal detachment. A subject suspected of having retinal detachment may also have one or more risk factors associated with retinal detachment (e.g., photoreceptor cell death). A “subject suspected of having retinal detachment” is sometimes diagnosed with retinal detachment and is sometimes found to not have retinal detachment.

As used herein, the term “subject diagnosed with retinal detachment” refers to a subject who has been tested and found to have retinal cells detached from the subadjacent retinal pigment epithelial cells. Retinal detachment may be diagnosed using any suitable method, including but not limited to, biopsy, imaging techniques, blood test, and the detection methods of the present invention.

As used herein, the term “photoreceptor cell apoptosis,” “photoreceptor cell death,” or similar terms, refer to ocular tissue wherein a portion (e.g., 0.01%, 1%, 5%, 10%, 25%, 50%, 80%, 90%, 99%, 99.999%) of photoreceptor cells are experiencing cell death. Risk factors for photoreceptor apoptosis include, but are not limited to, retinal insult (e.g., retinal detachment).

As used herein, the term “characterizing retinal detachment in a subject,” or similar terms, refer to the identification of one or more properties associated with retinal detachment. Examples of such properties include, but are not limited to, quantifying IL-6 activity within ocular tissue. Examples of IL-6 activity include, but are not limited to, IL-6 activity, JAK/STAT pathway activity, TGF-β pathway activity, Ahr pathway activity, STAT expression and/or activity, FLIP expression and/or activity, endothelin 2 expression and/or activity, ceruloplasmin expression and/or activity, lipocalin 2 expression and/or activity, serpin A3N expression and/or activity, and fibroblast growth factor receptor-1 expression and/or activity.

As used herein, the term “IL-6 protective period,” or similar terms, refer to a period following a retinal insult (e.g., retinal detachment) where IL-6 expression and/or activity is increased within the affected ocular tissue (e.g., retinal tissue and/or retinal pigment epithelia) thereby preventing photoreceptor apoptosis. In some embodiments, the IL-6 protective period gradually diminishes over time resulting in a gradual increase in photoreceptor apoptosis in the affected ocular tissue.

As used herein, the term “immunoglobulin” or “antibody” refer to proteins that bind a specific antigen. Immunoglobulins include, but are not limited to, polyclonal, monoclonal, chimeric, and humanized antibodies, Fab fragments, F(ab′)₂ fragments, and includes immunoglobulins of the following classes: IgG, IgA, IgM, IgD, IbE, and secreted immunoglobulins (sIg). Immunoglobulins generally comprise two identical heavy chains and two light chains. However, the terms “antibody” and “immunoglobulin” also encompass single chain antibodies and two chain antibodies.

As used herein, the term “antigen binding protein” refers to proteins that bind to a specific antigen. “Antigen binding proteins” include, but are not limited to, immunoglobulins, including polyclonal, monoclonal, chimeric, and humanized antibodies; Fab fragments, F(ab′)₂ fragments, and Fab expression libraries; and single chain antibodies.

The term “epitope” as used herein refers to that portion of an antigen that makes contact with a particular immunoglobulin.

When a protein or fragment of a protein is used to immunize a host animal, numerous regions of the protein may induce the production of antibodies which bind specifically to a given region or three-dimensional structure on the protein; these regions or structures are referred to as “antigenic determinants”. An antigenic determinant may compete with the intact antigen (i.e., the “immunogen” used to elicit the immune response) for binding to an antibody.

The terms “specific binding” or “specifically binding” when used in reference to the interaction of an antibody and a protein or peptide means that the interaction is dependent upon the presence of a particular structure (i.e., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabelled A) in a reaction containing labeled “A” and the antibody will reduce the amount of labeled A bound to the antibody.

As used herein, the terms “non-specific binding” and “background binding” when used in reference to the interaction of an antibody and a protein or peptide refer to an interaction that is not dependent on the presence of a particular structure (i.e., the antibody is binding to proteins in general rather that a particular structure such as an epitope).

As used herein, the term “specifically binding to IL-6, FLIP, STAT with low background binding,” or similar terms refer to an antibody that binds specifically to IL-6, STAT and/or FLIP protein (e.g., in an immunohistochemistry assay) but not to other proteins (e.g., lack of non-specific binding).

As used herein, the term “instructions for using said kit for detecting an ophthalmology disorder associated with photoreceptor cell death and/or retinal insult in said subject” includes instructions for using the reagents contained in the kit for the detection and characterization of such a disorder in a sample from a subject.

As used herein, the term “administration” refers to the act of giving a drug, prodrug, or other agent, or therapeutic treatment (e.g., compositions of the present invention) to a subject (e.g., a subject or in vivo, in vitro, or ex vivo cells, tissues, and organs). Exemplary routes of administration to the human body can be through the eyes (ophthalmic), intra-vitreal, peri-ocular, mouth (oral), skin (transdermal), nose (nasal), lungs (inhalant), oral mucosa (buccal), ear, by injection (e.g., intravenously, subcutaneously, intratumorally, intraperitoneally, etc.) and the like.

As used herein, the term “co-administration” refers to the administration of at least one agent(s) (e.g., exogenous IL-6) and one or more other agents—e.g., an agent designed to prevent and/or inhibit photoreceptor apoptosis) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s).

As used herein, the term “toxic” refers to any detrimental or harmful effects on a subject, a cell, or a tissue as compared to the same cell or tissue prior to the administration of the toxicant.

As used herein, the term “pharmaceutical composition” refers to the combination of an active agent (e.g., exogenous IL-6) with a carrier, inert or active, making the composition especially suitable for diagnostic or therapeutic use in vitro, in vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “topically” refers to application of the compositions of the present invention to the surface of the skin and mucosal cells and tissues (e.g., alveolar, buccal, lingual, masticatory, or nasal mucosa, and other tissues and cells that line hollow organs or body cavities).

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), and various types of wetting agents, any and all solvents, dispersion media, coatings, sodium lauryl sulfate, isotonic and absorption delaying agents, disintrigrants (e.g., potato starch or sodium starch glycolate), and the like. The compositions also can include stabilizers and preservatives. For examples of carriers, stabilizers and adjuvants. (See e.g., Martin, Remington's Pharmaceutical Sciences, 15th Ed., Mack Publ. Co., Easton, Pa. (1975), incorporated herein by reference in its entirety).

As used herein, the term “pharmaceutically acceptable salt” refers to any salt (e.g., obtained by reaction with an acid or a base) of a compound of the present invention that is physiologically tolerated in the target subject (e.g., a mammalian subject, and/or in vivo or ex vivo, cells, tissues, or organs). “Salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, sulfonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, chloride, bromide, iodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like. For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

### DETAILED DESCRIPTION OF THE INVENTION

Retinal detachment (RD), separation of the neurosensory retina from subjacent retinal pigment epithelium (RPE), causes photoreceptor cell death and retinal remodeling (see, e.g., Kroll A, et al., Am J Ophthalmol. 1968; 66(3):410-427; Fisher SK, et al., Prog Retin Eye Res. 2005; 24(3):395-431; Sethi C S, et al., Invest Ophthalmol Vis Sci. 2005; 46(1):329-342; each herein incorporated by reference in their entireties), which may lead to severe visual impairment (see, e.g., Burton T C, Trans Am Ophthalmol Soc. 1982; 80:475-497; Hassan, T S, et al., Ophthalmology. 2002; 109(1):146-152; each herein incorporated by reference in their entireties). If photoreceptors are separated long enough from their underlying source of metabolic support, apoptotic cell death ensues (see, e.g., Cook B, et al., Invest Ophthalmol Vis Sci. 1995; 36(6):990-996; Hisatomi T, et al., Curr Eye Res. 2002; 24(3):161-172; Zacks DN, et al., Invest Ophthalmol Vis Sci. 2003; 44(3):1262-1267; Yang, L., et al., Invest Ophthalmol Vis Sci. 2004; 45(2):648-654; each herein incorporated by reference in their entireties). However, in rodent and feline models of RD, the peak time of photoreceptor apoptosis, as measured with terminal deoxynucleotidyl transferase dUTP nick end labeling (TUNEL) staining, cell counts, and caspase activation, does not occur until 3 days after detachment (see, e.g., Cook B, et al., Invest Ophthalmol Vis Sci. 1995; 36(6):990-996; Zacks D N, et al., Invest Ophthalmol Vis Sci. 2003; 44(3):1262-1267; Yang, L., et al., Invest Ophthalmol Vis Sci. 2004; 45(2):648-654; each herein incorporated by reference in their entireties). Similarly, retrospective case series have demonstrated that patients with macula-off RDs that are repaired within 5 days after onset retained 20/20 vision (see, e.g., Burton T C. Trans Am Ophthalmol Soc. 1982; 80:475-497; herein incorporated by reference in its entirety) and 53-71% of those repaired within 9-10 days after onset retained 20/50 vision or better (see, e.g., Burton T C, Trans Am Ophthalmol Soc. 1982; 80:475-497; Hassan, T. S., Ophthalmology. 2002; 109(1):146-152; each herein incorporated by reference in their entireties). This delayed peak in photoreceptor apoptosis and visual loss after RD demonstrates a balance between neuroprotective and neurotoxic factors with neuroprotective factors prevailing initially, but neurotoxic factors predominating after longer periods of detachment.

Understanding what intrinsic neuroprotective mechanisms are initially activated after retinal detachment allows augmentation of those mechanisms to prevent photoreceptor cell death and visual loss after RD. Microarray analysis of experimental 24-hour detachments in rats revealed increased expression of genes involved in stress-response pathways, including the interleukin-6 (IL-6)/signal transducer and activator of transcription (STAT) pathway, tumor growth factor (TGF)-β pathway, and the aryl hydrocarbon (Ahr) pathway (see, e.g., Zacks D N, et al., Invest Ophthalmol Vis Sci. 2006; 47(4):1691-1695; herein incorporated by reference in its entirety). In situ hybridization studies of 4-day mouse RDs have demonstrated increased transcription of genes encoding endothelin 2, ceruloplasmin, lipocalin 2, and serpin A3N (see, e.g., Rattner A, et al., J Neurosci. 2005; 25(18):4540-4549; herein incorporated by reference in its entirety). Quantitative reverse transcriptase polymerase chain reaction (RT-PCR) of dissociated rat photoreceptors has shown upregulation of fibroblast growth factor receptor-1 (FGFR1) as early as 6 hours after RD (see, e.g., Ozaki S, et al., Invest Ophthalmol Vis Sci. 2000; 41(2):568-579; herein incorporated by reference in its entirety).

IL-6 is a pleotropic cytokine with a role in inflammation, hematopoiesis, angiogenesis, cell differentiation, and neuronal survival (see, e.g., John G R, et al., Neuroscientist. 2003; 9(1):10-22; Heinrich, P C, et al., Biochem J. 2003; 374(Pt 1):1-20; Ebrahem Q, et al., Invest Ophthalmol Vis Sci. 2006; 47(11):4935-4941; each herein incorporated by reference in their entireties). In terms of host response to a foreign pathogen, IL-6 has been shown, in mice, to be required for resistance against the bacterium, Streptococcus pneumoniae (see, e.g., van der Poll T, et al., 1997 J Infect Dis 176 (2): 439-44; herein incorporated by reference in its entirety). IL-6 is also a “myokine,” a cytokine produced from muscle, and is elevated in response to muscle contraction (see, e.g., Febbraio M A, et al., 2005 Exerc Sport Sci Rev 33 (3): 114-9; herein incorporated by reference in its entirety). Additionally, osteoblasts secrete IL-6 to stimulate osteoclast formation. Smooth muscle cells in the tunica media of many blood vessels also produce IL-6 as a pro-inflammatory cytokine.

IL-6 is a mediator of fever and of the acute phase response. In the muscle and fatty tissue IL-6 stimulates energy mobilization which leads to increased body temperature. IL-6 can be secreted by macrophages in response to specific microbial molecules, referred to as pathogen associated molecular patterns (PAMPs). These PAMPs bind to highly important detection molecules of the innate immune system, called Toll-like receptors (TLRs), that are present on the cell surface (or in intracellular compartments) which induce intracellular signaling cascades that give rise to inflammatory cytokine production. IL-6 is also essential for hybridoma growth and is found in many supplemental cloning media such as briclone. Inhibitors of IL-6 (including estrogen) are used to treat postmenopausal osteoporosis.

IL-6 signals through a cell-surface type I cytokine receptor complex consisting of the ligand-binding IL-6Rα chain (CD 126), and the signal-transducing component gp130 (also called CD130). CD 130 is the common signal transducer for several cytokines including leukemia inhibitory factor(LIF), ciliary neurotropic factor, oncostatin M, IL-11 and cardiotrophin-1, and is almost ubiquitously expressed in most tissues. In contrast, the expression of CD 126 is restricted to certain tissues. As IL-6 interacts with its receptor, it triggers the gp130 and IL-6R proteins to form a complex, thus activating the receptor. These complexes bring together the intracellular regions of gp130 to initiate a signal transduction cascade through certain transcription factors, Janus kinases (JAKs) and Signal Transducers and Activators of Transcription (STATs).

IL-6 is a well studied cytokine that uses gp130 in its signaling complex. Other cytokines that signal through receptors containing gp130 include, but are not limited to, Interleukin 11 (IL-11), Interleukin 27 (IL-27), ciliary neurotrophic factor (CNTF), cardiotrophin-1 (CT-1), cardiotrophin-like cytokine (CLC), leukemia inhibitory factor (LIF), oncostatin M (OSM), and Kaposi's sarcoma associated herpes virus interleukin 6 like protein (KSHV-IL6) (see, e.g., Kishimoto T, et al., 1995 Blood 86: 1243-1254; herein incorporated by reference in its entirety). These cytokines are commonly referred to as the IL-6 like or gp130 utilizing cytokines.

In addition to the membrane-bound receptor, a soluble form of IL-6R (sIL-6R) has been purified from human serum and urine. Many neuronal cells are unresponsive to stimulation by IL-6 alone, but differentiation and survival of neuronal cells can be mediated through the action of sIL-6R. The sIL-6R/IL-6 complex can stimulate neurites outgrowth promote survival of neurons, hence are important in nerve regeneration through remyelination.

In the central nervous system and retina, IL-6 is synthesized by microglia, astrocytes, and neurons (see, e.g., Gadient R A, et al., Prog Neurobiol. 1997; 52(5):379-390; Schobitz, B., et al., Eur J Neurosci. 1993; 5(11): 1426-1435; each herein incorporated by reference in their entireties). The neurotrophic activity of IL-6 results from, for example, IL-6 belonging to a family of neuropoietic cytokines which includes ciliary neurotrophic factor (CNTF) and leukemia inhibitory factor (LIF) (see, e.g., Heinrich, P C, et al., Biochem J. 2003; 374(Pt 1):1-20; herein incorporated by reference in its entirety). The receptors for these cytokines share the gp130 subunit. IL-6 binds to either the membrane bound-IL-6 receptor (IL-6R) or the soluble form of the receptor (sIL-6R). The sIL-6R can be secreted from cells allowing other cell types that do not normally express IL-6R to be sensitive to the IL-6 in a mechanism termed trans-signaling (see, e.g., Heinrich, P C, et al., Ann N Y Acad Sci. 1995; 762:222-236; herein incorporated by reference in its entirety). In rat models, IL-6 protein levels are upregulated approximately 8 hours after retinal ischemia reperfusion injury and 3 days after RD (see, e.g., Sanchez, R N, et al., Invest Ophthalmol Vis Sci. 2003; 44(9):4006-4011; Zacks D N, et al., Invest Ophthalmol Vis Sci. 2006; 47(4): 1691-1695; each herein incorporated by reference in their entireties). In vitro studies have shown that IL-6 increases the duration of rat retinal ganglion cell survival in primary tissue culture (see, e.g., Mendonca Torres, P M, et al., J Neuroimmunol. 2001; 117(1-2):43-50; Sappington, et al., Invest Ophthalmol Vis Sci. 2006; 47(7):2932-2942; each herein incorporated by reference in their entireties). Furthermore, intravitreal injection of exogenous IL-6 immediately after ischemia reperfusion injury or prior to N-methyl-D-aspartate (NMDA)-induced toxicity increases survival of retinal ganglion cells in rat models (see, e.g., Sanchez, R N, et al., Invest Ophthalmol Vis Sci. 2003; 44(9):4006-4011; Inomata, Y., et al., Biochem Biophys Res Commun. 2003; 302(2):226-232; each herein incorporated by reference in their entireties). Collectively, the data indicate that IL-6, IL-6 receptors, and/or related pathways are upregulated in the retina after injury as a neuronal survival factor.

With regard to the mechanism by which IL-6 acts as an anti-apoptotic factor, IL-6 is an activator of the Janus kinase (JAK)/signal transducer and activator of transcription (STAT) pathway (see, e.g., Samardzija, M., et al., FASEB J. 2006; 20(13):2411-2413; Heinrich, P C, et al., Biochem J. 2003; 374(Pt 1): 1-20; each herein incorporated by reference in their entireties). IL-6 potently activates STAT3, and to a lesser extent, STATI (see, e.g., Briscoe, J., et al., Curr Biol. 1994; 4(11):1033-1035; Gerhartz, C., et al., J Biol Chem. 1996; 271(22):12991-12998; Heinrich, P C, et al., Biochem J. 2003; 374(Pt 1): 1-20; each herein incorporated by reference in their entireties). In general, STAT1 is associated with tumor suppression and pro-apoptotic activity whereas STAT3 is predominantly associated with cellular proliferation and considered to be anti-apoptotic (see, e.g., Samardzija, M., et al., FASEB J. 2006; 20(13):2411-2413; Aaronson D S, et al., Science. 2002; 296(5573):1653-1655; Stephanou A, et al., Int J Exp Pathol. 2003; 84(6):239-244; Stephanou A, et al., Growth Factors. 2005; 23(3):177-182; Battle T E, et al., Curr Mol Med. 2002; 2(4):381-392; each herein incorporated by reference in their entireties). Modulation of apoptotic pathways by STATs occurs, for example, though upregulation of factors that trigger cell death, such as FAS and caspases, or promote cell survival, such as Bcl-xL and FLICE (FADD (Fas-associated death domain)-like interleukin-1β-converting enzyme) inhibitory protein (FLIP) (see, e.g., Battle T E, et al., Curr Mol Med. 2002; 2(4):381-392; Haga, S., J Clin Invest. 2003; 112(7):989-998; each herein incorporated by reference in their entireties). FLIP is an enzymatically inactive homologue of caspase-8 that can compete with caspase-8 for recruitment to death-inducing signaling complexes (DISCs), and thereby acts as, for example, a dominant negative inhibitor of apoptosis (see, e.g., Budd, R C, et al., Nat Rev Immunol. 2006; 6(3):196-204; herein incorporated by reference in its entirety). As IL-6 stabilizes protein levels of FLIP, FLIP is more rapidly degraded in IL-6^{-/-} mice (see, e.g., Kovalovich, K., et al., J Biol Chem. 2001; 276(28):26605-26613; herein incorporated by reference in its entirety).

Experiments conducted during the development of embodiments for the present invention examined the role of IL-6 following RD and subsequent photoreceptor cell death and retinal remodeling. It was shown that inhibition of IL-6 accelerates photoreceptor apoptosis in rodent models of RD. It was also shown that the presence of IL-6 expression within a subretinal space following an event resulting in RD hindered (e.g., prevented, diminished, reduced) photoreceptor apoptosis. In addition, it was shown that exogenous administration of IL-6 into the subretinal space prevented photoreceptor apoptosis in rodent models of RD. It was also shown that photoreceptor apoptosis following an event involving the retina (e.g., RD) is initially prevented through expression of, for example, IL-6, IL-6 receptor activity, and/or pathway related activity (“the IL-6 protective period”). The IL-6 protective period, however, diminishes over time as IL-6 activity diminishes, thereby resulting in a gradual increase in photoreceptor apoptosis.

Accordingly, the present invention relates to compositions and methods for the detecting, treating, and researching ophthalmic disorders associated with photoreceptor cell death and/or retinal insult (e.g., RD). In particular, the present invention provides compositions and methods for modulating (e.g., reducing) photoreceptor cell death following retinal detachment through, for example, increasing IL-6 expression and/or activity (e.g., exogenous IL-6), activating IL-6 receptors (e.g., IL-6R, sIL6-R), activating pathway related compounds (e.g., STAT, FLIP), and/or activating related pathways (e.g., JAK/STAT pathway, TGFβ pathway, Ahr pathway). In addition, the present invention provides IL-6 (e.g., exogenous IL-6), IL-6 receptors (e.g., IL-6R, sIL6-R), and/or pathway related compounds (e.g., STAT, FLIP) for the diagnosis, treatment, and empirical investigation of ophthalmic disorders associated with photoreceptor cell death and/or retinal insult (e.g., RD). Exemplary compositions and methods of the present invention are described in more detail in the following sections: I. Detection of Opthalmic Disorders; II. In vivo Imaging; III. Antibodies; IV. Therapeutics; V. Pharmaceutical Compositions; VI. Drug Screening; and VII. Kits.

The practice of the present invention employs, unless otherwise indicated, conventional techniques of organic chemistry, pharmacology, molecular biology (including recombinant techniques), cell biology, biochemistry, and immunology, which are within the skill of the art. Such techniques are explained fully in the literature, such as, “Molecular cloning: a laboratory manual” Second Edition (Sambrook et al., 1989); “Oligonucleotide synthesis” (M. J. Gait, ed., 1984); “Animal cell culture” (R. I. Freshney, ed., 1987); the series “Methods in enzymology” (Academic Press, Inc.); “Handbook of experimental immunology” (D. M. Weir & C. C. Blackwell, eds.); “Gene transfer vectors for mammalian cells” (J. M. Miller & M. P. Calos, eds., 1987); “Current protocols in molecular biology” (F. M. Ausubel et al., eds., 1987, and periodic updates); “PCR: the polymerase chain reaction” (Mullis et al., eds., 1994); and “Current protocols in immunology” (J. E. Coligan et al., eds., 1991), each of which is incorporated herein by reference in their entireties.

### I. Detection of Ophthalmic Disorders

The present invention provides methods of detecting ophthalmic disorders associated with photoreceptor cell death and/or retinal insult. Examples of such disorders include, but are not limited to, RD, retinal artery occlusion, disorders involving rhegmatogenous retinal detachment (e.g., posterior vitreous detachment, peripheral retinal lesions (e.g., enclosed oral bays, meridional folds, cystic retinal tufts, lattice degeneration), myopia, senile retinoschisis, cataract extraction, retinal trauma, intraocular inflammation/infection, acute retinal necrosis syndrome, cytomegalovirus retinitis, ocular toxocariasis, ocular toxoplasmosis, pars planitis, colobomas of the choroid and retina, coloboma of the lens (e.g., giant retinal tear), Stickler syndrome, Goldmann-Favre syndrome, Marfan syndrome, homocystinuria, Ehlers-Danlos syndrome), disorders involving tractional retinal detachment (e.g., proliferative vitreoretinopathy, proliferative diabetic retinopathy, sickle cell (SC) disease (e. g., hemoglobin SC, hemoglobin S-thalassemia), familial exudative vitreoretinopathy, retinopathy of prematurity, penetrating trauma with vitreous bands, cataract surgery with vitreous loss), disorders involving exudative retinal detachment (e.g., primary tumors (e.g., malignant melanoma of the choroid, hemangioma of the choroid, retinoblastoma), metastatic carcinoma to the choroid (e.g., breast cancer, lung cancer), inflammation (e.g., choroiditis [Harada disease], retinitis [toxoplasmosis, CMV]), vascular disease (e.g., angiomatosis of the retina (e.g., Von Hippel disease), telangiectasia retina, juvenile coat disease, adult coat disease, Eales disease, retinal vein occlusion), disorders involving optic nerve disease (e.g., pit of the optic disc with serous detachment of the macula, nerve head drusen with serosanguineous detachment of adjacent retina, Leber Stellate maculopathy), disorders involving macular disease (e.g., central serous chorioretinopathy, age-related macular degeneration, other causes of disciform detachment, ocular histoplasmosis, angioid streaks, high myopia (>6 diopters)), systemic diseases (e.g., toxemia, uremia, systemic lupus erythematosus [SLE], leukemia), lesions that may simulate retinal detachment (e.g., cerebrovascular accident or transient ischemic attack, optic neuritis, atypical migraine, vitreous (e.g., membranes, hemorrhages, inflammation), posterior vitreous detachment, retinal lesions (e.g., primary retinoschisis, juvenile retinoschisis, degenerative retinoschisis, secondary retinoschisis, retinopathy of prematurity, diabetic retinopathy, retinal artery occlusion (mainly branch retinal artery occlusion), and/or disorders involving choroidal detachment (e.g., serous, hemorrhagic).

In certain embodiments, the present invention provides methods for detecting ophthalmic disorders associated with photoreceptor cell death and/or retinal insult involving, for example, detecting and quantifying IL-6 expression in an affected ocular region (e.g., an ocular region experiencing photoreceptor cell death and/or retinal detachment).

Experiments conducted during the development of embodiments for the present invention demonstrated that inhibition of IL-6 accelerates photoreceptor apoptosis in models of RD. As such, in some embodiments, detection of diminished (e.g., reduced, absent) IL-6 expression, IL-6 receptor activity (e.g., IL-6R, sIL6-R), and/or related pathway activity (e.g., FLIP expression, STAT expression) in an affected ocular region (e.g., RPE tissue, retinal tissue) following an ocular insult (e.g., retinal detachment) indicates an increased likelihood of photoreceptor cell death and potential visual impairment. In some embodiments wherein retinal detachment has occurred, detection of diminished (e.g., reduced, absent) IL-6 expression, IL-6 receptor activity (e.g., IL-6R, sIL6-R), and/or related pathway activity (e.g., FLIP expression, STAT expression) in the affected ocular region (e.g., RPE tissue, retinal tissue) indicates a diminishing of the IL-6 protective period following retinal insult and a likelihood of photoreceptor cell death and visual impairment. In addition, experiments conducted during the development of embodiments for the present invention demonstrated that the presence of IL-6 within the subretinal space following an event involving the retina (e.g., retinal trauma resulting in RD) prevented photoreceptor apoptosis in models of RD. As such, in some embodiments wherein retinal detachment has occurred, detection of IL-6 expression, IL-6 receptor activity (e.g., IL-6R, sIL6-R), and/or related pathway activity (e.g., FLIP expression, STAT expression) in the affected ocular region (e.g., RPE tissue, retinal tissue) indicates the likelihood that photoreceptor cell death is being hindered (e.g., prevented, reduced) by the presence of such IL-6 activity. In some embodiments wherein retinal detachment has occurred, detection of IL-6 expression, IL-6 receptor activity (e.g., IL-6R, sIL6-R), and/or related pathway activity (e.g., FLIP expression, STAT expression) in the affected ocular region (e.g., RPE tissue, retinal tissue) indicates the likelihood that the IL-6 protective period is in effect. In some embodiments, detection of a change (e.g., a reduction) in IL-6 expression, IL-6 receptor activity (e.g., IL-6R, sIL6-R), and/or related pathway activity (e.g., FLIP expression, STAT expression) between at least two time points in an affected ocular region (e.g., an area of retinal detachment) indicates a diminishing of the IL-6 protective period, and an increased risk for photoreceptor cell death and potential visual impairment.

The present invention provides IL-6 expression and/or related pathway activity (e.g., FLIP expression, STAT expression) in an affected ocular region (e.g., an area of retinal detachment) as photoreceptor cell death biomarkers. In some embodiments, the present invention provides methods for detecting and quantifying expression of photoreceptor cell death biomarkers (e.g., IL-6, FLIP, STAT). In some embodiments, expression is measured directly (e.g., at the nucleic acid level). In some embodiments, expression is detected in tissue samples (e.g., retinal tissue, RPE tissue). The present invention further provides panels and kits for the detection of photoreceptor cell death biomarkers (e.g., IL-6, FLIP, STAT). In some embodiments, the presence of photoreceptor cell death biomarker expression over a period of time (e.g., between two time points, three time points, ten time points, etc) is used to provide a prognosis to a subject regarding photoreceptor apoptosis and/or visual impairment. For example, detection of reduced IL-6 expression between two time periods in an affected ocular region (e.g., an area involving RD) indicates a diminishment and/or ceasement of the IL-6 protective period and an increased risk for photoreceptor cell death and/or visual impairment. For example, detection of increased IL-6 expression between two time periods in an affected ocular region (e.g., an area involving RD) indicates the presence of an IL-6 protective period and the unlikelihood of immediate photoreceptor cell death and visual impairment. In some embodiments, comparing expression of photoreceptor cell death biomarkers (e.g., IL-6, FLIP, STAT) over a period of time may be used to test the efficacy of a treatment (e.g., drugs directed toward treating RD) and/or may be used to test the efficacy of a new form of treatment (e.g., new drugs directed toward treating RD; surgical procedures directed toward treating RD).

In some embodiments, detection of the presence or absence of ophthalmic disorders associated with photoreceptor cell death and/or retinal insult (e.g., RD) is accomplished through comparing expression levels of photoreceptor cell death biomarkers (e.g., IL-6, FLIP, STAT) within the retina and/or subadjacent retinal pigment epithelium (RPE) to established thresholds. For example, in some embodiments, a subject's stage of RD is accomplished through comparing expression levels of the subject's photoreceptor cell death biomarkers (e.g., IL-6, STAT, FLIP) with established photoreceptor cell death biomarker threshold levels for ophthalmic disorders associated with photoreceptor cell death and/or retinal insult (e.g., established IL-6 threshold level for the IL-6 protective period; established IL-6 threshold level for increased risk for photoreceptor cell death; established IL-6 threshold level for medium risk for photoreceptor cell death; established IL-6 threshold level for low photoreceptor cell death risk). Established threshold levels may be generated from any number of sources, including but not limited to, groups of subjects (e.g., adult male and female humans) having RD with and/or without a treatment, groups of subjects not having RD, groups of subjects having photoreceptor cell death, groups of subjects not having photoreceptor cell death. Any number of subjects within a group may be used to generate an established threshold (e.g., 5 subjects, 10 subjects, 20, 30, 50, 500, 5000, 10,000, etc.).

The information provided through detection of photoreceptor cell death biomarkers (e.g., IL-6, FLIP, STAT) within retinal tissue and/or RPE tissue can also be used to direct a course of treatment (e.g., a treatment designed to prevent photoreceptor cell death while repairing the retina for such subjects experiencing photoreceptor cell death; a treatment designed to repair the retina without concern for photoreceptor cell death for such subjects not yet experiencing photoreceptor cell death). Moreover, if a subject's photoreceptor cell death biomarker levels indicate a high risk for photoreceptor cell death, exogenous IL-6 may be administered to prevent such photoreceptor cell death.

In some embodiments, detection of photoreceptor cell death biomarkers (e.g., IL-6, FLIP, STAT) is accomplished, for example, by measuring the levels of IL-6, IL-6 receptor activity, and/or pathway related compounds (e.g., FLIP, STAT) in cells and tissue (e.g., retinal tissue, RPE tissue). For example, in some embodiments, IL-6 can be monitored using antibodies directed toward IL-6, FLIP, and/or STAT. In some embodiments, detection is performed on cells or tissue (e.g., retinal tissue, RPE tissue) after the cells or tissues are removed from the subject. In other embodiments, detection is performed by visualizing the photoreceptor cell death biomarker (e.g., IL-6, FLIP, STAT) in cells and tissues (e.g., retinal tissue, RPE tissue) residing within a subject.

In some embodiments, detection of photoreceptor cell death biomarkers (e.g., IL-6, FLIP, STAT) is accomplished by measuring the accumulation of corresponding mRNA in a tissue sample (e.g., retinal tissue, RPE tissue). mRNA expression may be measured by any suitable method known in the art.

In some embodiments, detection of photoreceptor cell death biomarkers (e.g., IL-6, FLIP, STAT) is accomplished through protein expression. Protein expression may be detected by any suitable method. In some embodiments, proteins are detected by binding of an antibody specific for the protein. The present invention is not limited to a particular antibody. Any antibody (monoclonal or polyclonal) that specifically detects photoreceptor cell death biomarkers (e.g., IL-6, FLIP, STAT) may be utilized. In some embodiments, photoreceptor cell death biomarkers (e.g., IL-6, FLIP, STAT) are detected by immunohistochemistry. In other embodiments, photoreceptor cell death biomarkers (e.g., IL-6, FLIP, STAT) are detected by their binding to an antibody raised against photoreceptor cell death biomarkers. In some embodiments, commercial antibodies directed toward photoreceptor cell death biomarkers (e.g., IL-6, FLIP, STAT) are utilized.

Antibody binding is detected by techniques known in the art (e.g., radioimmunoassay, ELISA (enzyme-linked immunosorbant assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, etc.), complement fixation assays, immunofluorescence assays, protein A assays, and immunoelectrophoresis assays, etc.

In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many methods are known in the art for detecting binding in an immunoassay and are within the scope of the present invention.

In some embodiments, an automated detection assay is utilized. Methods for the automation of immunoassays include those described in U.S. Pat. Nos. 5,885,530, 4,981,785, 6,159,750, and 5,358,691, each of which is herein incorporated by reference. In some embodiments, the analysis and presentation of results is also automated.

In other embodiments, the immunoassay is as described in U.S. Pat. Nos. 5,599,677 and 5,672,480; each of which is herein incorporated by reference.

### II. In Vivo Imaging

In some embodiments, in vivo imaging techniques are used to visualize and quantify the expression of photoreceptor cell death biomarkers (e.g., IL-6, FLIP, STAT) in an animal (e.g., a human or non-human mammal). For example, in some embodiments, photoreceptor cell death biomarker mRNA or protein is labeled using a labeled antibody specific for the biomarker. Specifically bound and labeled antibodies can be quantified and detected in an individual using any in vivo imaging method, including, but not limited to, radionuclide imaging, positron emission tomography, computerized axial tomography, X-ray or magnetic resonance imaging method, fluorescence detection, and chemiluminescent detection.

The in vivo imaging methods of the present invention are useful in the research use and the diagnosis of ophthalmic disorders associated with photoreceptor cell death and/or retinal insult (e.g., RD) in cells that contain the biomarkers of the present invention (e.g., retinal tissue and/or RPE tissue). In vivo imaging is used to quantify and visualize the presence of a biomarker indicative of an ophthalmic disorders associated with photoreceptor cell death and/or retinal insult (e.g., RD). Such techniques allow for diagnosis without the use of a biopsy. In some embodiments, the in vivo imaging methods of the present invention are useful for providing prognoses to patients (e.g., patients suffering from RD). For example, the presence of photoreceptor cell death biomarkers (e.g., IL-6, FLIP, STAT) expressed below a certain threshold may be indicative of an increased risk for photoreceptor cell death and/or visual impairment. The presence of photoreceptor cell death biomarkers (e.g., IL-6, FLIP, STAT) expressed below a certain threshold may be indicative of an increased likelihood for response to a particular treatment form (e.g., administration of exogenous IL-6).

In some embodiments, reagents (e.g., antibodies) specific for the biomarkers of the present invention are fluorescently labeled. The labeled antibodies can be introduced into a subject (e.g., orally or parenterally). Fluorescently labeled antibodies are detected using any suitable method (e.g., using the apparatus described in U.S. Pat. No. 6,198,107, herein incorporated by reference).

In other embodiments, antibodies are radioactively labeled. The use of antibodies for in vivo diagnosis is well known in the art. Sumerdon et al., (Nucl. Med. Biol 17:247-254 (1990) have described an optimized antibody-chelator for the radioimmunoscintographic imaging of tumors using Indium-111 as the label. Griffin et al., (J Clin Onc 9:631-640 (1991)) have described the use of this agent in detecting tumors in patients suspected of having recurrent colorectal cancer, each herein incorporated by reference in their entirety. The use of similar agents with paramagnetic ions as labels for magnetic resonance imaging is known in the art (See, e.g., Lauffer, Magnetic Resonance in Medicine 22:339-342 (1991), herein incorporated by reference in its entirety). The label used will depend on the imaging modality chosen. Radioactive labels such as Indium-111, Technetium-99m, or Iodine-131 can be used for planar scans or single photon emission computed tomography (SPECT). Positron emitting labels such as Fluorine-19 can also be used for positron emission tomography (PET). For MRI, paramagnetic ions such as Gadolinium (III) or Manganese (II) can be used.

Radioactive metals with half-lives ranging from 1 hour to 3.5 days are available for conjugation to antibodies, such as scandium-47 (3.5 days) gallium-67 (2.8 days), gallium-68 (68 minutes), technetiium-99m (6 hours), and indium-111 (3.2 days), of which gallium-67, technetium-99m, and indium-111 are preferable for gamma camera imaging, gallium-68 is preferable for positron emission tomography.

A useful method of labeling antibodies with such radiometals is by means of a bifunctional chelating agent, such as diethylenetriaminepentaacetic acid (DTPA), as described, for example, by Khaw et al. (Science 209:295 (1980)) for In-111 and Tc-99m, and by Scheinberg et al. (Science 215:1511 (1982), each herein incorporated by reference in their entirety. Other chelating agents may also be used, but the 1-(p-carboxymethoxybenzyl)EDTA and the carboxycarbonic anhydride of DTPA are advantageous because their use permits conjugation without affecting the antibody's immunoreactivity substantially.

Another method for coupling DPTA to proteins is by use of the cyclic anhydride of DTPA, as described by Hnatowich et al. (Int. J. Appl. Radiat. Isot. 33:327 (1982)), herein incorporated by reference in its entirety, for labeling of albumin with In-111, but which can be adapted for labeling of antibodies. A suitable method of labeling antibodies with Tc-99m which does not use chelation with DPTA is the pretinning method of Crockford et al., (U.S. Pat. No. 4,323,546, herein incorporated by reference).

One method of labeling immunoglobulins with Tc-99m is that described by Wong et al. (Int. J. Appl. Radiat. Isot., 29:251 (1978)), herein incorporated by reference in its entirety, for plasma protein, and recently applied successfully by Wong et al (J. Nucl. Med., 23:229 (1981)), herein incorporated by reference in its entirety, for labeling antibodies.

In the case of the radiometals conjugated to the specific antibody, it is likewise desirable to introduce as high a proportion of the radiolabel as possible into the antibody molecule without destroying its immunospecificity. A further improvement may be achieved by effecting radiolabeling in the presence of the specific biomarker of the present invention, to insure that the antigen binding site on the antibody will be protected. The antigen is separated after labeling.

In still further embodiments, in vivo biophotonic imaging (Xenogen, Almeda, Calif.) is utilized for in vivo imaging. This real-time in vivo imaging utilizes luciferase. The luciferase gene is incorporated into cells, microorganisms, and animals (e.g., as a fusion protein with a biomarker of the present invention). When active, it leads to a reaction that emits light. A CCD camera and software can be used to capture the image and analyze it.

### III. Antibodies

The present invention provides isolated antibodies. In some embodiments, the present invention provides monoclonal antibodies that specifically bind to the photoreceptor cell death biomarkers (e.g., IL-6, FLIP, STAT). Examples include, but are not limited to, monoclonal and polyclonal antibodies against IL-6 (Abcam #Ab6672, Abcam #Ab21515, Abcam #Ab17506, Abcam #Ab7746, Abcam #Ab9626, Abcam #Ab11449, Abcam #Ab14038, Abcam #Ab43142, Abcam #Ab17529, Abcam #Ab32530), monoclonal and polyclonal antibodies against FLIP (Abcam #Ab16078, Abcam #Ab8423, Abcam #Ab4042, Abcam #Ab15319, Abcam #Ab6144, Abcam #Ab21486, Abcam #Ab8421, Abcam #Ab10864, Abcam #Ab13577, Abcam #Ab9945, Abcam #Ab13683), and monoclonal and polyclonal antibodies against STAT (Abcam #Ab30646, Abcam #Ab5073, Abcam #Ab52906, Abcam #Ab52630, Abcam #Ab996, Abcam #Ab32500, Abcam #Ab32043, Abcam #Ab32520, Abcam #Ab4742, Abcam #Ab31369). These antibodies, and others, find use in the diagnostic and therapeutic methods described herein.

An antibody against a biomarker of the present invention may be any monoclonal or polyclonal antibody, as long as it can recognize the biomarker. Antibodies can be produced by using a biomarker of the present invention as the antigen according to a conventional antibody or antiserum preparation process.

### IV. Therapeutics

In some embodiments, the present invention provides a method of treating or researching ophthalmic disorders associated with photoreceptor cell death and/or retinal insult. Examples of such disorders include, but are not limited to, RD, retinal artery occlusion, disorders involving rhegmatogenous retinal detachment (e.g., posterior vitreous detachment, peripheral retinal lesions (e.g., enclosed oral bays, meridional folds, cystic retinal tufts, lattice degeneration), myopia, senile retinoschisis, cataract extraction, retinal trauma, intraocular inflammation/infection, acute retinal necrosis syndrome, cytomegalovirus retinitis, ocular toxocariasis, ocular toxoplasmosis, pars planitis, colobomas of the choroid and retina, coloboma of the lens (e.g., giant retinal tear), Stickler syndrome, Goldmann-Favre syndrome, Marfan syndrome, homocystinuria, Ehlers-Danlos syndrome), disorders involving tractional retinal detachment (e.g., proliferative vitreoretinopathy, proliferative diabetic retinopathy, sickle cell (SC) disease (e. g., hemoglobin SC, hemoglobin S-thalassemia), familial exudative vitreoretinopathy, retinopathy of prematurity, penetrating trauma with vitreous bands, cataract surgery with vitreous loss), disorders involving exudative retinal detachment (e.g., primary tumors (e.g., malignant melanoma of the choroid, hemangioma of the choroid, retinoblastoma), metastatic carcinoma to the choroid (e.g., breast cancer, lung cancer), inflammation (e.g., choroiditis [Harada disease], retinitis [toxoplasmosis, CMV]), vascular disease (e.g., angiomatosis of the retina (e.g., Von Hippel disease), telangiectasia retina, juvenile coat disease, adult coat disease, Eales disease, retinal vein occlusion), disorders involving optic nerve disease (e.g., pit of the optic disc with serous detachment of the macula, nerve head drusen with serosanguineous detachment of adjacent retina, Leber Stellate maculopathy), disorders involving macular disease (e.g., central serous chorioretinopathy, age-related macular degeneration, other causes of disciform detachment, ocular histoplasmosis, angioid streaks, high myopia (>6 diopters)), systemic diseases (e.g., toxemia, uremia, systemic lupus erythematosus [SLE], leukemia), lesions that may simulate retinal detachment (e.g., cerebrovascular accident or transient ischemic attack, optic neuritis, atypical migraine, vitreous (e.g., membranes, hemorrhages, inflammation), posterior vitreous detachment, retinal lesions (e.g., primary retinoschisis, juvenile retinoschisis, degenerative retinoschisis, secondary retinoschisis, retinopathy of prematurity, diabetic retinopathy, retinal artery occlusion (mainly branch retinal artery occlusion), and/or disorders involving choroidal detachment (e.g., serous, hemorrhagic).

The present invention is not limited to particular methods for treating such disorders. Experiments conducted during the development of embodiments for the present invention demonstrated that inhibition of IL-6 accelerates photoreceptor apoptosis in rodent models of RD, that the presence of IL-6 expression within a subretinal space following an event resulting in RD hindered (e.g., prevented, diminished, reduced) photoreceptor apoptosis, that exogenous administration of IL-6 into the subretinal space prevented photoreceptor apoptosis in rodent models of RD, that photoreceptor apoptosis following an event involving the retina (e.g., RD) was initially prevented through expression of, for example, IL-6, IL-6 receptor activity, and/or pathway related activity (“the IL-6 protective period”), and that the IL-6 protective period, however, diminished over time as IL-6 activity diminished, thereby resulting in a gradual increase in photoreceptor apoptosis. Accordingly, in some embodiments, the methods comprise altering (e.g., reducing, inhibiting) photoreceptor cell death through increasing and/or retaining IL-6 expression, IL-6 receptor activity (e.g., IL-6R, sIL6-R), and/or related pathway activity (e.g., FLIP expression, STAT expression) in an affected ocular region (e.g., an area of retinal detachment).

The present invention is not limited to particular techniques and/or methods for increasing and/or retaining IL-6 expression, IL-6 receptor activity (e.g., IL-6R, sIL6-R), and/or related pathway activity (e.g., FLIP expression, STAT expression) in an affected ocular region (e.g., an area of retinal detachment). In some embodiments, increasing and/or retaining IL-6 expression, IL-6 receptor activity (e.g., IL-6R, sIL6-R), and/or related pathway activity (e.g., FLIP expression, STAT expression) in an affected ocular region (e.g., an area of retinal detachment) is accomplished through administration of an agent (e.g., a pharmaceutical agent) designed to increase IL-6 expression, IL-6 receptor activity (e.g., IL-6R, sIL6-R), and/or related pathway activity (e.g., FLIP expression, STAT expression) in an affected ocular region (e.g., an area of retinal detachment). The present invention is not limited to a particular type of pharmaceutical agent designed to increase IL-6 expression, IL-6 receptor activity (e.g., IL-6R, sIL6-R), and/or related pathway activity (e.g., FLIP expression, STAT expression) in an affected ocular region (e.g., an area of retinal detachment).

In some embodiments, exogenous IL-6 is administered for purposes of increasing and/or retaining IL-6 expression, IL-6 receptor activity (e.g., IL-6R, sIL6-R), and/or related pathway activity (e.g., FLIP expression, STAT expression) in an affected ocular region (e.g., an area of retinal detachment). The methods of the present invention are not limited to a particular method or technique for administering exogenous IL-6. In some embodiments, exogenous IL-6 is administered orally and/or parenterally. In some embodiments, IL-6 is directly applied (e.g., surgically) to the area of interest (e.g., the retina and/or RPE tissue region). The methods are not limited to administration of a particular amount of exogenous IL-6. In some embodiments, the amount of exogenous IL-6 administered is sufficient to prevent photoreceptor cell death for a desired amount of time (e.g., a minute, an hour, a day, a week, etc.).

In some embodiments, the methods of the present invention further comprise co-administration of an additional agent designed to treat ophthalmic disorders involving photoreceptor cell death, retinal detachment, and/or retinal degeneration. Examples of such additional agents include, but are not limited to, drugs designed to treat ophthalmic disorders involving photoreceptor cell death, retinal detachment, and/or retinal degeneration. In situ hybridization studies of 4-day mouse RDs have demonstrated increased transcription of genes encoding endothelin 2, ceruloplasmin, lipocalin 2, and serpin A3N (see, e.g., Rattner A, et al., J Neurosci. 2005; 25(18):4540-4549; herein incorporated by reference in its entirety). Quantitative reverse transcriptase polymerase chain reaction (RT-PCR) of dissociated rat photoreceptors has shown upregulation of fibroblast growth factor receptor-1 (FGFR1) as early as 6 hours after RD (see, e.g., Ozaki S, et al., Invest Ophthalmol Vis Sci. 2000; 41(2):568-579; herein incorporated by reference in its entirety). Accordingly, in some embodiments, the agents are designed to increase expression and/or activity of one or more of endothelin 2, ceruloplasmin, lipocalin 2, serpin A3N, and FGFR1.

In some embodiments, the methods of the present invention further comprise co-administration of one or more general ophthalmological pharmacological agents. Examples of general pharmacological agents include, but are not limited to, Lucentis (ranibizumab), Macugen (pegaptanib); Restasis (cyclosporine ophthalmic emulsion); Lumigan (bimatoprost ophthalmic solution); Travatan (travoprost ophthalmic solution); Valcyte (valganciclovir HCl); Betaxon; Quixin (levofloxacin); Rescula (unoprostone isopropyl ophthalmic solution) 0.15%; Visudyne (verteporfin for injection); Alamast; Zaditor; Alrex; Cosopt; Lotemax; Salagen Tablets; Viroptic; Vitravene Injection; Acular; Acular (ketorolac tromethamine ophthalmic solution) 0.5%; BSS Sterile Irrigating Solution; AK-Con-A (naphazoline ophthalmic); Alphagan (brimonidine); Ocuflox (ofloxacin opthalmic solution) 0.3%; OcuHist; Vistide (cidofovir injection); and Vitrasert Implant.

In some embodiments, the methods of the present invention further comprise co-administration of an additional procedure designed to treat ophthalmic disorders involving photoreceptor cell death, retinal detachment, and/or retinal degeneration. Examples of such procedures include, but are not limited to, cryopexy and laser photocoagulation, scleral buckle surgery, pneumatic retinopexy, and vitrectomy.

Furthermore, any of the therapies described herein can be tested and developed in animal models (e.g., rats, mice, cats, pigs, cows, primates).

### V. Pharmaceutical Compositions

The present invention further provides pharmaceutical compositions (e.g., comprising an exogenous IL-6, and/or agents designed to increase IL-6 expression). The pharmaceutical compositions of the present invention may be administered in a number of ways depending upon whether local or systemic treatment is desired and upon the area to be treated. Administration may be topical, pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

In some embodiments, the invention provide pharmaceutical compositions containing (a) one or more agents designed to increase IL-6 expression and/or activity and (b) one or more other agents designed to treat ophthalmic disorders involving photoreceptor cell death, retinal detachment, and/or retinal degeneration.

Dosing may be dependent on severity and responsiveness of the disease state (e.g., stage of RD) to be treated, with the course of treatment lasting from several minutes, hours, and/or days to several months, or until photoreceptor cell death is sufficiently prevented. Optimal dosing schedules can be calculated from measurements of drug accumulation in the body of the patient. The administering physician can easily determine optimum dosages, dosing methodologies and repetition rates. Optimum dosages may vary depending on the relative potency of individual oligonucleotides, and can generally be estimated based on EC₅₀s found to be effective in in vitro and in vivo animal models or based on the examples described herein. In general, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the treatment (e.g., exogenous IL-6) is administered in maintenance doses, ranging from 0.01 μg to 100 g per kg of body weight, once or more daily, to once every, for example, hour, day, week and/or month.

### VI. Drug Screening

In some embodiments, the present invention provides drug screening assays (e.g., to screen for new drugs for treating ophthalmic disorders associated with photoreceptor cell death and/or retinal insult). The screening methods of the present invention utilize photoreceptor cell death biomarkers (e.g., IL-6, FLIP, STAT) identified using the methods of the present invention. For example, in some embodiments, the present invention provides methods of screening for compounds that alter (e.g., increase or decrease), directly or indirectly, the presence of photoreceptor cell death biomarkers (e.g., IL-6, FLIP, STAT). In some embodiments, candidate compounds are antisense agents (e.g., siRNAs, oligonucleotides, etc.) directed against IL-6, STAT, and/or FLIP. In other embodiments, candidate compounds are antibodies that specifically bind to a photoreceptor cell death biomarker (e.g., IL-6, FLIP, STAT) of the present invention. Also contemplated to be discoverable using the compositions and methods of the present invention are proteins, peptides, peptide mimetics, small molecules and other agents that can be used to treat ophthalmic disorders involving photoreceptor cell death, retinal detachment, and/or retinal degeneration.

In one screening method, candidate compounds are evaluated for their ability (e.g., increase) to alter biomarker presence, activity or expression by contacting a compound with a cell (e.g., a retinal cell and/or RPE cell) and then assaying for the effect of the candidate compounds on the presence or expression of a photoreceptor cell death biomarker (e.g., IL-6, FLIP, STATIn some embodiments, the effect of candidate compounds on expression or presence of a photoreceptor cell death biomarker (e.g., IL-6, FLIP, STAT) is assayed for by detecting the level of biomarker present within the cell. In other embodiments, the effect of candidate compounds on expression or presence of a biomarker is assayed for by detecting the level of photoreceptor cell death biomarker (e.g., IL-6, FLIP, STAT) present in the extracellular matrix.

In other embodiments, the effect of candidate compounds on expression or presence of biomarkers is assayed by measuring the level of polypeptide encoded by the biomarkers. The level of polypeptide expressed can be measured using any suitable method, including but not limited to, those disclosed herein.

The test compounds of the present invention can be obtained using any of the numerous approaches in combinatorial library methods known in the art, including biological libraries; peptoid libraries (libraries of molecules having the functionalities of peptides, but with a novel, non-peptide backbone, which are resistant to enzymatic degradation but which nevertheless remain bioactive; see, e.g., Zuckennann et al., J. Med. Chem. 37: 2678-85 (1994), herein incorporated by reference in its entirety); spatially addressable parallel solid phase or solution phase libraries; synthetic library methods requiring deconvolution; the ‘one-bead one-compound’ library method; and synthetic library methods using affinity chromatography selection. The biological library and peptoid library approaches are preferred for use with peptide libraries, while the other four approaches are applicable to peptide, non-peptide oligomer or small molecule libraries of compounds (Lam (1997) Anticancer Drug Des. 12:145, herein incorporated by reference in its entirety).

Examples of methods for the synthesis of molecular libraries can be found in the art, for example in: DeWitt et al., Proc. Natl. Acad. Sci. U.S.A. 90:6909 (1993); Erb et al., Proc. Nad. Acad. Sci. USA 91:11422 (1994); Zuckermann et al., J. Med. Chem. 37:2678 (1994); Cho et al., Science 261:1303 (1993); Carrell etal., Angew. Chem. Int. Ed. Engl. 33.2059 (1994); Carell et al., Angew. Chem. Int. Ed. Engl. 33:2061 (1994); and Gallop et al., J. Med. Chem. 37:1233 (1994), each herein incorporated by reference in their entirety.

Libraries of compounds may be presented in solution (e.g., Houghten, Biotechniques 13:412-421 (1992)), or on beads (Lam, Nature 354:82-84 (1991), herein incorporated by reference in its entirety), chips (Fodor, Nature 364:555-556 (1993), herein incorporated by reference in its entirety), bacteria or spores (U.S. Pat. No. 5,223,409; herein incorporated by reference), plasmids (Cull et al., Proc. Nad. Acad. Sci. USA 89:18651869 (1992), herein incorporated by reference in its entirety) or on phage (Scott and Smith, Science 249:386-390 (1990); Devlin Science 249:404-406 (1990); Cwirla et al., Proc. Natl. Acad. Sci. 87:6378-6382 (1990); Felici, J. Mol. Biol. 222:301 (1991), each herein incorporated by reference in their entirety).

This invention further pertains to novel agents identified by the above-described screening assays. Accordingly, it is within the scope of this invention to further use an agent identified as described herein (e.g., a biomarker modulating agent, a biomarker specific antibody, or a biomarker-binding substrate) in an appropriate animal model (such as those described herein) to determine the efficacy, toxicity, side effects, or mechanism of action, of treatment with such an agent. Furthermore, novel agents identified by the above-described screening assays can be, e.g., used for treatments as described herein.

### VII. Kits

In yet other embodiments, the present invention provides kits for the detection, characterization, and/or treatment of ophthalmic disorders associated with photoreceptor cell death and/or retinal insult (e.g., RD). In some embodiments, the kits contain exogenous IL-6. In some embodiments, the kits contain antibodies specific for photoreceptor cell death biomarkers (e.g., IL-6, FLIP, STAT). In some embodiments, the kits further contain detection reagents and buffers. In other embodiments, the kits contain reagents specific for the detection of nucleic acids (e.g., DNA, RNA, mRNA or cDNA, oligonucleotide probes or primers). In some embodiments, the kits contain all of the components necessary and/or sufficient to perform a detection assay, including all controls, directions for performing assays, and any necessary software for analysis and presentation of results.

### EXPERIMENTAL

The following examples are provided in order to demonstrate and further illustrate certain some embodiments and aspects of the present invention and are not to be construed as limiting the scope thereof.

### EXAMPLE I

This example describes the materials and methods used for Example II.

Retinal Detachments Retinal detachments were created in adult male Brown-Norway rats (300-400 g), wild-type C57 mice, and IL-6^{-/-} mice (see, e.g., Zacks D N, et al., Invest Ophthalmol Vis Sci. 2006; 47(4): 1691-1695; herein incorporated by reference in its entirety). Briefly, rodents were anesthetized with a 50:50 mix of ketamine (100 mg/mL) and xylazine (20 mg/mL), and pupils were dilated with topical phenylephrine (2.5%) and tropicamide (1%). A 20-gauge microvitreoretinal blade (Walcott Scientific, Marmora, N.J.) was used to create a sclerotomy 2 mm posterior to the limbus, carefully avoiding lens damage. A Glaser subretinal injector (32-gauge tip; BD Ophthalmic Systems, Sarasota, Fla.) was introduced through the sclerotomy into the vitreous cavity and then through a peripheral retinotomy into the subretinal space. Sodium hyaluronate (10 mg/mL) (Healon; Pharmacia and Upjohn Co., Kalamazoo, Mich.) was slowly injected to detach the neurosensory retina from the underlying retinal pigment epithelium. In all experiments, approximately one third to one half of the retina was detached. Detachments were created in the left eye, leaving the right eye as the control. In some eyes, either 0.1 μg anti-rat IL-6 neutralizing antibody (R&D Systems, Minneapolis, Minn.), 0.15 μg anti-human IL-6 neutralizing antibody (R&D Systems, Minneapolis, Minn.), or 15 ng exogenous human IL-6 (R&D Systems, Minneapolis, Minn.) was injected into the subretinal space of the detachment in a 10 μl volume at the time of creation of the detachment or at a time point following creation of the detachment.

Histology and Terminal Deoxynucleotidyl Transferase dUTP Nick End Labeling (TUNEL) Staining At varying intervals after creation of the detachment, the animals were euthanized, and the eyes were enucleated. For TUNEL staining, whole eyes were fixed overnight at 4° C. in phosphate-buffered saline with 4% paraformaldehyde (pH 7.4). The specimens were embedded in paraffin and sectioned at a thickness of 5-6 μm. TUNEL staining was performed on the sections with the APOPTAG Fluorescein In Situ Apoptosis Detection Kit according to the manufacturer's instructions (Millipore, Billerica, Mass.). Photoreceptor cell apoptosis was quantified by counting both surviving and TUNEL positive cells in the outer nuclear layer (ONL) and expressed as the percentage of total cells in the ONL that were TUNEL positive. For light microscopic analysis, the eyes were fixed overnight at 4° C. in 0.1 M cacodylate buffer with 2.5% glutaraldehyde and 2% formaldehyde (pH 7.4). Samples were then post-fixed in 2% osmium textroxide, dehydrated in graded alcohol, and embedded in epoxy resin and sectioned at a thickness of 5-6 μm. Sections were stained with 0.5% toluidine blue in 0.1% borate buffer. The number of cells in the ONL, the thickness of the ONL, and the thickness of the total retina were measured.

For quantification, the data from three non-overlapping sections was averaged for each eye unless there were less than three non-overlapping sections in which case fewer sections were used. Statistical analysis comparing percentage of TUNEL positive cells in the ONL between groups and comparing the ONL cell count/total retinal thickness ratio between groups was performed using 2-tailed Student's t test without assuming equal variance. Differences were considered significant at P<0.05.

Western Blot Analysis Retinas from experimental eyes with detachments and control eyes without detachments were dissected from the RPE-choroid at 3 days after retinal detachment, homogenized, and lysed in buffer containing 10 mM HEPES (pH 7.6), 0.5% IgEPal, 42 mM KCl, 1 mM phenylmethylsulfonyl fluoride (PMSF), 1 mM EDTA, 1 mM EGTA, 1 mM dithiothreitol (DTT), and 5 mM MgCl₂ and 1 tablet of protease inhibitors per 10 mL buffer (Complete Mini; Roche Diagnostics GmbH, Mannheim, Germany). The homogenates were incubated on ice and centrifuged at 22,000×g at 4° C. for 60 minutes. The protein concentration of the supernatant was then determined (DC Protein Assay kit; Bio-Rad Laboratories, Hercules Calif.). The protein samples were loaded and run on SDS-polyacrylamide gels (4%-20% Tris-HCl Ready Gels; Bio-Rad Laboratories). After electrophoretic separation, the proteins were transferred onto polyvinylidene fluoride (PVDF) membranes (Immobilon-P; Amersham Pharmacia Biotech, Piscataway, N.J.). Protein bands were visualized with Ponceau S staining and the lanes assessed for equal loading by densitometry of a nonspecific band present across all lanes. Membranes were then placed in 5% nonfat powdered milk in TBS (150 mM NaCl, and 50 mM Tris [pH 7.6]) and incubated overnight at 4° C. on a shaker. Membranes were then incubated with the primary antibody in 2.5% powdered milk in TBS overnight at 4° C. Membranes were washed extensively with TBS-T (0.1% Tween-20) and incubated with horseradish-peroxidase-labeled secondary antibody (1:3000; Santa Cruz Biotechnology, Santa Cruz, Calif.) for 1 hour at room temperature. Bands were visualized by chemiluminescence (ECL-Plus; Amersham Pharmacia Biotech) according to the manufacturer's instructions. Antibodies against the following proteins were used: phospho-STAT1 (Tyr701) or phospho-STAT3 (Tyr705) using an immunoblotting kit (PhosphoPlus Statl [Tyr701] Antibody Kit 9170 or PhosphoPlus Stat3 [Tyr705] Antibody Kit 9130, respectively; Cell Signaling Technology, Danvers, Mass.) according to the manufacturer's instructions using a 1:1000 dilution of the primary antibody.

### EXAMPLE II

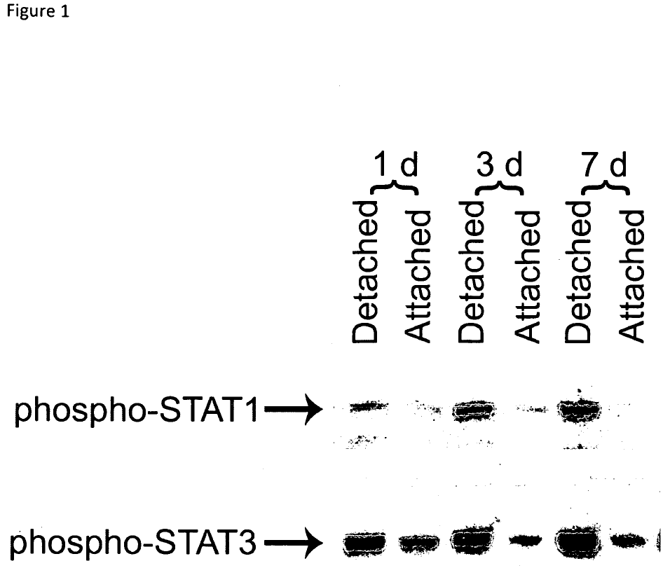

In the context of retinal-RPE separation, it has been shown that STAT1 and STAT3 transcript and protein levels are increased (see, e.g., Zacks DN, et al., Invest Ophthalmol Vis Sci. 2003; 44(3): 1262-1267; herein incorporated by reference in its entirety). FIG. 1 demonstrates increased phosphorylation (e.g.,, activation) of STAT1 and STAT3 in detached retinas compared with attached retinas (see, e.g., Chong D Y, et al., Invest Ophthalmol Vis Sci. 2008; 49(7)3193-3200; herein incorporated by reference in its entirety). Injection of the IL-6-neutralizing antibody into the subretinal space at the time of detachment resulted in approximately a 50% reduction in the level of phosphorylated STAT3 (see, e.g., Chong D Y, et al., Invest Ophthalmol Vis Sci. 2008; 49(7)3193-3200; herein incorporated by reference in its entirety). There was not, however, any reduction in the level of phosphorylated STAT1. These data show that after retinal-RPE separation, the IL-6 effect is mediated predominantly through STAT3 but not STAT1.

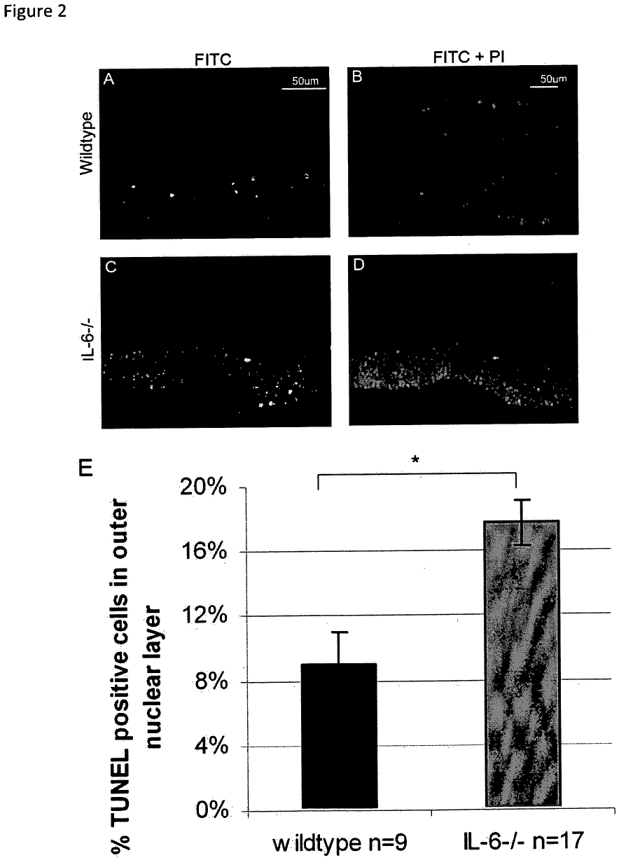

To determine the role performed by IL-6 in inhibition apoptosis of rat retinal photoreceptors after RD, RDs were created in wild-type C57 and IL-6^{-/-} mice. Three days after detachment, the eyes were harvested, and apoptosis within the retina was evaluated with TUNEL staining (FIG. 2). TUNEL positive cells were confined to the ONL of photoreceptors, consistent with prior studies of experimental RD (see, e.g., Zacks D N, et al., Invest Ophthalmol Vis Sci. 2003; 44(3):1262-1267; Chong D Y, et al., Invest Ophthalmol Vis Sci. 2008; 49(7)3193-3200; each herein incorporated by reference in their entirety). As shown in FIG. 2, the percentage of TUNEL positive cells in the ONL was significantly greater in the wild-type C57 mice compared to the IL-6^{-/-} mice (9% vs. 18%, respectively, P=0.002).

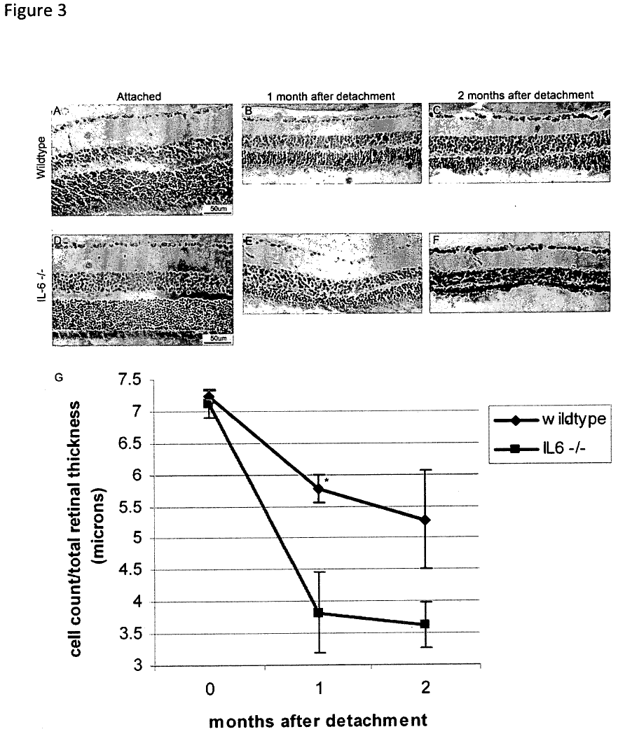

To investigate the effect of genetic ablation of IL-6 on longer term changes in retinal morphology after RD, detachments in wild-type C57 and IL-6^{-/-} mice were created and maintained for 1 and 2 months, and eyes were harvested for toluidine blue staining. The number of photoreceptor cells in the ONL per high power field normalized to the thickness of the entire retina from nerve fiber layer to ONL in the high power field was measured as a marker of photoreceptor loss (FIG. 3; Zacks DN, et al., Invest Ophthalmol Vis Sci. 2003; 44(3):1262-1267; Chong D Y, et al., Invest Ophthalmol Vis Sci. 2008; 49(7)3193-3200; each herein incorporated by reference in their entirety). There was a large decline in the ONL cell count at the 1 month time point as compared to attached retinas at the zero time point for both wild-type and IL-6^{-/-} mice, but the ONL cell count/total retinal thickness was significantly lesser in the IL-6^{-/-} mice as compared to the wild-type mice at the 1 month time point (3.82 vs. 5.78, respectively, P=0.02) (see FIG. 3).

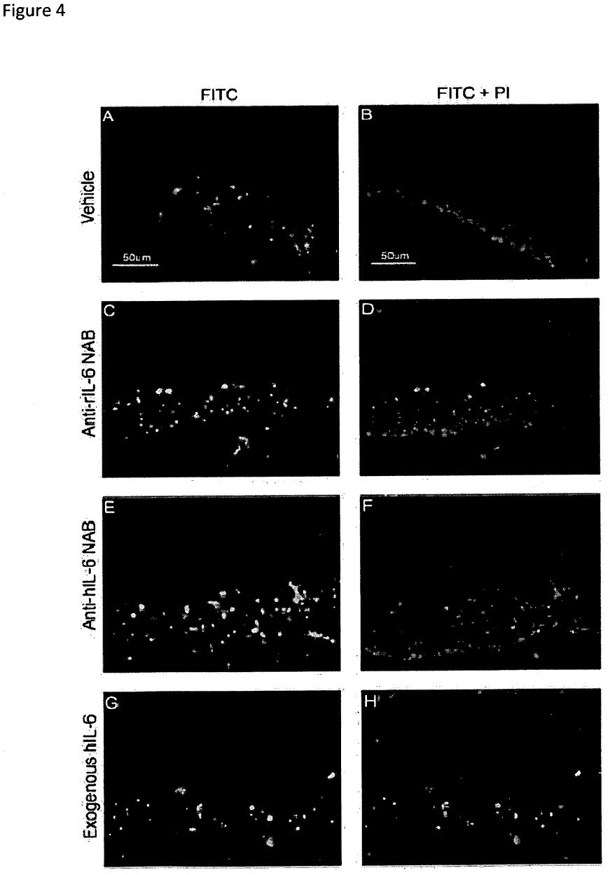

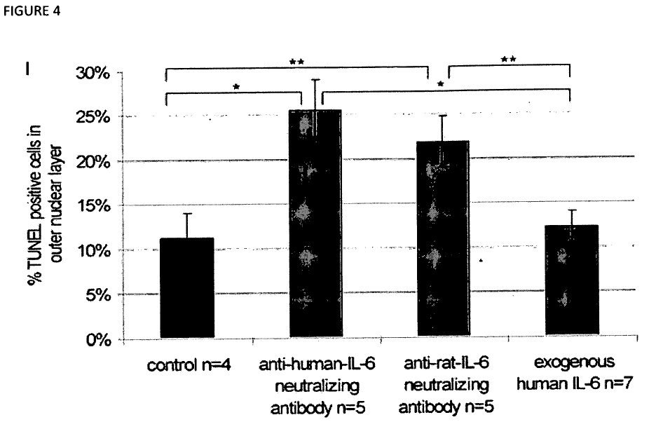

To further support the data from IL-6^{-/-} mice that that loss of IL-6 activity increases photoreceptor apoptosis after RD, RD detachments were created in Brown Norway rats and either vehicle only or vehicle plus 0.1 g anti-rat IL-6 (rIL-6) neutralizing antibody (NAB) or 0.15 μg anti-human IL-6 (hIL-6) NAB was injected subretinally at the time of detachment. TUNEL staining of rat eyes 3 days after detachment revealed significantly higher percentages of TUNEL positive cells in the ONL of retinas treated with anti-rIL-6 NAB (22%, P=0.03 anti-rIL-6 NAB vs. vehicle control) or anti-hIL-6 NAB (26%, P=0.01 anti-hIL-6 NAB vs. vehicle control) compared to vehicle only (11%) (see, FIGS. 4A-F and I; Zacks D N, et al., Invest Ophthalmol Vis Sci. 2003; 44(3):1262-1267; Chong D Y, et al., Invest Ophthalmol Vis Sci. 2008; 49(7)3193-3200; each herein incorporated by reference in their entirety). To see if administering exogenous IL-6 would protect photoreceptors from RD-induced apoptosis, 15 ng recombinant hIL-6 was injected subretinally at the time of creation of the detachment, and eyes were harvested for TUNEL staining 3 days after retinal detachment. Interestingly, the percentage of TUNEL positive cells in the group of rats with subretinal injection of exogenous hIL-6 was no different than that of rats treated with vehicle alone (12% vs. 11%) (see, FIGS. 4G-H and I).

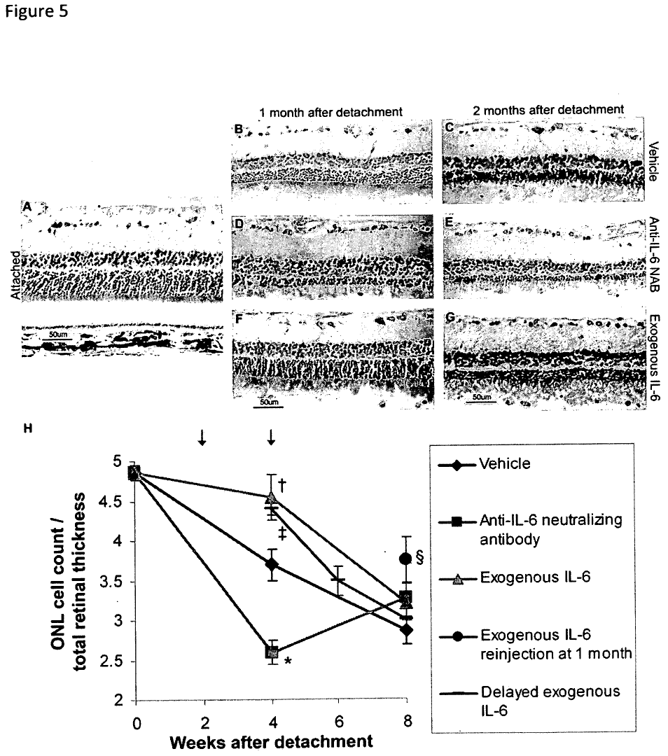

In detachments carried out to 1 and 2 months, subretinal injection of anti-hIL-6 NAB at the time of detachment resulted in significantly lower normalized ONL cell counts 1 month after detachment compared to subretinal injection of vehicle only (P=0.001; FIGS. 5B, 5F, 5H;) (see, e.g., Chong D Y, et al., Invest Ophthalmol Vis Sci. 2008; 49(7)3193-3200; herein incorporated by reference in its entirety). In contrast, subretinal administration of exogenous hIL-6 at the time of detachment resulted in significantly higher ONL cell counts/total retinal thickness ratios 1 month after detachment compared to controls injected with vehicle only (P=0.05; see FIGS. 5B, 5F, 5H). Subretinal administration of exogenous hIL-6 appeared to slow the rate of photoreceptor cell loss during the first month after detachment, but the rate accelerated during the second month after detachment such that the protective benefit of exogenous hIL-6 was lost, and the ONL cell count/total retinal thickness ratios were similar between groups treated with vehicle only, anti-hIL-6 NAB, and exogenous hIL-6 groups 2 months after detachment (see FIGS. 5C, 5E, 5G, 5H).

To determine whether the protective effect of exogenous IL-6 seen at 4 weeks could be extended by a second subretinal injection of exogenous IL-6 at that time, rats were injected with exogenous IL-6 at the time of creation of the detachment followed by a second injection of the same dose of exogenous IL-6 at 4 weeks after detachment. As shown, at 8 weeks after creation of the retinal detachment, the ONL cell count/total retinal thickness was still significantly higher in animals with repeat IL-6 injection at 4 weeks than in control animals (P=0.02; FIG. 5H). To determine whether delaying subretinal administration of IL-6 by 2 weeks after creation of the detachment could still prevent or delay photoreceptor apoptosis, the retinal-RPE separation was created, followed, 2 weeks after creation of the detachment, by the injection of exogenous IL-6. Eyes were harvested 4, 6, and 8 weeks after detachment (e.g., 2, 4, and 6 weeks after subretinal IL-6 injection), respectively, and stained with toluidine blue. As shown, the ONL cell count/total retinal thickness was minimally lower in the group, in which IL-6 injection was delayed 2 weeks compared with the group in which IL-6 injection was administered at the time of creation of the detachment (P=0.07), and it was significantly higher than the control animals (P=0.02; FIG. 5H).

All publications and patents mentioned in the above specification are herein incorporated by reference. Various modifications and variations of the described compositions and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

We claim:

1. A method of preventing photoreceptor apoptosis, comprising
providing ocular tissue comprising photoreceptor cells at risk for undergoing cellular apoptosis, and a composition configured to promote IL-6 activity within said ocular tissue; and
administering said composition to said ocular tissue, wherein said promotion of IL-6 activity prevents said photoreceptor cells from undergoing cellular apoptosis.

2. The method of claim 1, wherein said ocular tissue comprises tissue selected from the group consisting of retina and retinal pigment epithelium.

3. The method of claim 1, wherein said ocular tissue is within a human subject.

4. The method of claim 1, wherein said promotion of IL-6 activity comprises promotion of IL-6 expression within said ocular tissue.

5. The method of claim 1, wherein said promotion of IL-6 activity within said ocular tissue comprises promotion of activity of pathways selected from the group consisting of JAK/STAT, TGF-β, and Ahr.

6. The method of claim 1, wherein said promotion of IL-6 activity within said ocular tissue comprises promotion of expression of proteins selected from the group consisting of STAT1, STAT3, FLIP, endothelin 2, ceruloplasmin, lipocalin, serpin A3N, and fibroblast growth factor.

7. The method of claim 1, wherein said composition comprises exogenous IL-6.

8. The method of claim 1, wherein said ocular tissue comprises retinal tissue and retinal pigment epithelial tissue, wherein said retinal tissue is detached from said retinal pigment epithelial tissue.

9. A method of treating photoreceptor apoptosis, comprising
providing a subject having ocular tissue experiencing photoreceptor apoptosis, and a pharmaceutical composition configured to promote IL-6 activity within said ocular tissue; and
administering said pharmaceutical composition to said subject, wherein said administration inhibits said photoreceptor apoptosis within said ocular tissue.

10. The method of claim 9, wherein said ocular tissue comprises tissue selected from the group consisting of retina and retinal pigment epithelium.

11. The method of claim 9, wherein said ocular tissue comprises retinal tissue and retinal pigment epithelial tissue, wherein said retinal tissue is detached from said retinal pigment epithelial tissue.

12. The method of claim 9, wherein said subject is a human.

13. The method of claim 9, wherein said promotion of IL-6 activity comprises promotion of IL-6 expression within said ocular tissue.

14. The method of claim 9, wherein said promotion of IL-6 activity within said ocular tissue comprises promotion of activity of pathways selected from the group consisting of JAK/STAT, TGF-β, and Ahr.

15. The method of claim 9, wherein said promotion of IL-6 activity in said ocular tissue comprises promotion of expression of proteins selected from the group consisting of STAT1, STAT3, FLIP, endothelin 2, ceruloplasmin, lipocalin, serpin A3N, and fibroblast growth factor.

16. The method of claim 9, wherein said pharmaceutical composition comprises exogenous IL-6.

17. The method of claim 9, wherein said subject has been diagnosed with retinal detachment.

18. A method of identifying a photoreceptor apoptosis inhibitor, comprising providing ocular tissue comprising photoreceptor cells experiencing cellular apoptosis, and a composition comprising an agent;
administering said composition to said ocular tissue,
quantifying the amount of IL-6 activity and photoreceptor apoptosis in said ocular tissue following administration of said composition to said ocular tissue, wherein the presence of IL-6 activity and decreased photoreceptor apoptosis within said ocular tissue indicates said agent is a photoreceptor apoptosis inhibitor, wherein the absence of IL-6 activity and photoreceptor apoptosis within said ocular tissue indicates said agent is not a photoreceptor apoptosis inhibitor.

19. The method of claim 19, wherein said IL-6 activity comprises IL-6 expression within said ocular tissue.

20. The method of claim 19, wherein said IL-6 activity within said ocular tissue comprises activity of pathways selected from the group consisting of JAK/STAT, TGF-β, and Ahr.

21. The method of claim 19, wherein said IL-6 activity within said ocular tissue comprises expression of proteins selected from the group consisting of STAT1, STAT3, FLIP, endothelin 2, ceruloplasmin, lipocalin, serpin A3N, and fibroblast growth factor.
